(19) United States
(12) Patent Application Publication
(10) Pub. No.: US 2019/0209830 A1
(43) Pub. Date: Jul. 11, 2019

(54) TREATMENT OF VERTIGO USING ELECTRICAL STIMULATION

(71) Applicant: ReliefBand Technologies, LLC, Horsham, PA (US)

(72) Inventors: Nicholas Spring, Jenkintown, PA (US); Jacqueline Simon, New Hope, PA (US)

(21) Appl. No.: 16/241,018

(22) Filed: Jan. 7, 2019

Related U.S. Application Data
(60) Provisional application No. 62/614,212, filed on Jan. 5, 2018.
(60) Provisional application No. 62/614,215, filed on Jan. 5, 2018.

(51) Int. Cl. A61N 1/04; A61N 1/36; A61H 39/00

(52) U.S. Cl. CPC A61N 1/0456; A61N 1/36025; A61N 1/0492; A61N 1/044; A61N 1/3603; A61H 39/002

(57) ABSTRACT

A method of reducing or eliminating vertigo symptoms in an individual experiencing vertigo or an individual identified as being susceptible to vertigo is disclosed. The method includes positioning a non-invasive nerve stimulation device on the individual's wrist, wherein the electrodes of the non-invasive nerve stimulation device are in electrically conductive contact with skin at acupuncture point P6 of the wrist. The methods also include generating a stimulation signal and delivering the stimulation signal to the acupuncture point P6 of the wrist to prevent, reduce or eliminate one or more vertigo symptoms in the individual. The vertigo symptoms may include a feeling of spinning, tilting, swaying, or being unbalanced or pulled to one direction, vomiting, abnormal or jerking eye movements (nystagmus), headache, sweating, ringing in the ears, hearing loss, visual disturbances, weakness, difficulty speaking, a decreased level of consciousness, and difficulty walking.

### CROSS-REFERENCE TO RELATED APPLICATIONS

The present application claims priority to U.S. Provisional Patent Application Ser. No. 62/614,215, filed on Jan. 5, 2018, and U.S. Provisional Patent Application Ser. No. 62/614,212, filed on Jan. 5, 2018, the disclosures of which are hereby incorporated in their entirety at least by reference.

### FIELD OF THE INVENTION

The methods and devices described below relate to the fields of prevention and treatment of vertigo and vertigo symptoms using noninvasive electrical stimulation of an acupuncture point.

### BACKGROUND OF THE INVENTION

Vertigo is a feeling experienced by an individual in which the individual feels that they or their surroundings are moving or spinning when there is no actual movement. The motion commonly is described as a feeling of spinning or whirling, but it also can include sensations of falling, tilting, rotation, rocking or feeling off balance. Vertigo can cause nausea, vomiting, difficulties walking, standing, and maintaining balance. Vertigo can result in an individual falling.

Vertigo differs from dizziness in that vertigo describes an illusion of movement. Persons experiencing vertigo may feel like they are spinning or that the world around them is spinning despite in each case being perfectly still. Subjective vertigo refers to the condition in which a person experiencing vertigo feels as if they themselves are moving, it is called. Objective vertigo refers to the condition in which a person experiencing vertigo perceives that their surroundings or environment are moving is called objective vertigo. Vertigo often lasts for many hours or even days before resolving.

Vertigo can be caused by problems in the brain or spinal cord/central nervous system (central vertigo) but is often caused by an inner ear problem (peripheral vertigo). Vertigo is a symptom of other conditions and is not in itself contagious. There are several common causes of vertigo as well as some less common causes.

Benign paroxysmal positional vertigo, referred to as BPPV, is the most common form of vertigo. BPPV is characterized by the brief sensation of motion lasting 15 seconds to a few minutes. Such episodes are often described as a sudden attack of vertigo and may be initiated by sudden head movements or moving the head in a certain direction, such as rolling over in bed. BPPV may occur when tiny crystals and stones, calcium particles (canaliths), clump up in canals of the inner ear and may become displaced, causing irritation of the hair cells in the canals of the inner ear.

Vertigo may be caused by inflammation within the inner ear (labyrinthitis or vestibular neuritis), which is characterized by the sudden onset of vertigo and may also be accompanied by hearing loss. Viral and bacterial inner ear infections are often the most common cause of labyrinthitis. Examples of viruses that may cause labyrinthitis or vestibular neuritis include herpes viruses, influenza, measles, rubella, mumps, polio, hepatitis, and Epstein-Barr virus (EBV). The duration of symptoms can last for days until the inflammation subsides.

Meniere's disease is an inner ear disorder thought to be caused by a buildup of fluid and changing pressure in the ear, viral infections of the inner ear, head injury, a hereditary factor, or allergies. Common symptoms of is composed of Meniere's disease include vertigo, tinnitus and hearing loss. People with this condition have the abrupt onset of severe vertigo and fluctuating hearing loss as well as periods in which they are symptom-free.

Vertigo may be associated with head or neck injury, stroke or brain tumor, side effects from certain medications that cause ear damage and migraine headaches among other causes. Acoustic neuroma is an uncommon cause of vertigo related to a type of tumor of the nerve tissue of the inner ear.

Bertolucci, Nausea Control Device, U.S. Pat. No. 4,981,146, Jan. 1, 1991, which is incorporated herein by reference, describes a nausea control device in the form of a watch-like housing attachable to the human wrist by an adjustable attachment band. The device uses non -invasive nerve stimulation whereby electricity is passed through two electrodes to stimulate nerves located on the ventral side of the wrist (this anatomical position is sometimes referred to as the palmar side of the wrist). The ventral site of application is referred to as the P6 point, pericardium 6 point, or master point of the pericardium meridian (sometimes referred to as the vascular meridian). A primary object of the invention is to provide a non-chemical, non-invasive, painless and inexpensive method of alleviating nausea. It is also portable, self-contained and convenient to the patient, Electrical pulse repetition rate of approximately 70 pulses per second and a pulse width of 80 microseconds has been found to provide effective relief of nausea in a patient. Electrical pulse pattern comprises about 350 microsecond pulse width at about 31 pulses per second at power levels of about 8-40 milli-amps peak pulse height are disclosed. A wide range of pulse patterns may be used in non-invasive nerve stimulation devices. Nausea control devices similar to those described in U.S. Pat. No. 4,981,146 have been sold to treat nausea caused by chemotherapy, motion sickness and morning sickness associated with pregnancy and are used as an adjunct to antiemetic therapy in post-operative patients. Similar devices have been disclosed for use in treating dysmenorrhea and menstrual cramps (U.S. Pat. Nos. 6,282,443 and 6,718,202, which are each incorporated herein by reference), for use in moderating blood pressure (U.S. Pat. Nos. 6,178,352 and 6,658,298, which are each incorporated herein by reference) and for reducing gag reflex (U.S. Pat. No. 6,192,889, which is incorporated herein by reference).

### SUMMARY OF THE INVENTION

The method described below employs use of the device such as those described in U.S. Pat. Nos. 4,981,146, 6,076,018, 6,567,695, 6,735,480, 7,127,288, 7,171,266, 7,171,276, 7,983,761, provisional application provisional application No. 62/614,212 entitled WEARABLE DEVICE FOR DELIVERING ELECTRICAL STIMULATION TO THE P6 ACUPUNCTURE POINT and filed Jan. 5, 2017, which are each incorporated herein by reference, and similar devices for the relief and alleviation of symptoms associated with vertigo.

A method of reducing or eliminating vertigo symptoms in an individual experiencing vertigo is provided. The method may include the steps of: positioning a non-invasive nerve stimulation device on the individual's wrist, wherein the electrodes of the non-invasive nerve stimulation device are in electrically conductive contact with skin at acupuncture point P6 of the wrist; generating a. stimulation signal; and delivering the stimulation signal to the P6 acupuncture point of the wrist to reduce or eliminate one or more vertigo symptoms in the individual; wherein the vertigo symptoms are selected from the group consisting of: feeling of spinning, tilting, swaying, or being unbalanced or pulled to one direction, vomiting, abnormal or jerking eye movements (nystagmus), headache, sweating, ringing in the ears, hearing loss, visual disturbances, weakness, difficulty speaking, a decreased level of consciousness, and difficulty walking.

A method of preventing or reducing vertigo symptoms in an individual identified as being susceptible to vertigo is also provided. The method comprises the steps of: positioning a non-invasive nerve stimulation device on the individual's wrist, wherein the electrodes of the non-invasive nerve stimulation device are in electrically conductive contact with skin at acupuncture point P6 of the wrist; generating a stimulation signal; and delivering the stimulation signal to the acupuncture point P6 of the wrist to reduce or eliminate one or more vertigo symptoms in the individual. The vertigo symptoms may be selected from the group consisting of: feeling of spinning, tilting, swaying, or being unbalanced or pulled to one direction, vomiting, abnormal or jerking eye movements (nystagmus), headache, sweating, ringing in the ears, hearing loss, visual disturbances, weakness, difficulty speaking, a decreased level of consciousness, and difficulty walking.

In some embodiments of the method of reducing or eliminating vertigo symptoms in an individual experiencing vertigo and in some embodiments of the method of preventing or reducing vertigo symptoms in an individual identified as being susceptible to vertigo, the stimulation signal comprises individual pulses at power levels of about 1-100 milliamps peak pulse height, at a pulse frequency of about 31 Hz +/−10% such that there is a period of 32 msec between alternating monophasic pulses. In some embodiments, the stimulation signal comprises individual pulses at power levels of about 1-50 milliamps peak pulse height, or about 1-40 milliamps peak pulse height, or about 8-40 milliamps peak pulse height at tolerances of ±1.5%. In some embodiments, the stimulation signal comprises individual pulses at power levels selected from the group consisting of: about 8 milliamps peak pulse height at tolerance of ±15%; about 11 milliamps peak pulse height at tolerance of ±15%; about 14 milliamps peak pulse height at tolerance of ±15%; about 17 milliamps peak pulse height at tolerance of ±15%; about 20 milliamps peak pulse height at tolerance of ±15%; about 24 milliamps peak pulse height at tolerance of ±15%, about 28 milliamps peak pulse height at tolerance of ±15%; about 32 milliamps peak pulse height at tolerance of ±15%; about 36 milliamps peak pulse height at tolerance of ±15%; and about 40 milliamps peak pulse height at tolerance of ±15%. In some embodiments, the stimulation signal is delivered for 5-10 minutes or longer, or 30 minutes or longer, or 60 minutes or longer, or 90 minutes or longer, or 120 minutes or longer, or 150 minutes or longer, or 180 minutes or longer, or for longer than 180 minutes.

### DETAILED DESCRIPTION OF PREFERRED EMBODIMENTS

The biological effects of electrical stimulation of the P6 acupuncture point are more expansive than the suppression of nausea described in the Bertolucci patent. The methods described herein employ electrical stimulation of the P6 acupuncture point and may be used to reduce or eliminate nausea and one or more vertigo symptoms in addition to nausea. Symptoms of vertigo have been described as feeling as if one is spinning, tilting, swaying, or being unbalanced or pulled to one direction. Other symptoms that may be associated with vertigo include feeling nauseated, vomiting, abnormal or jerking eye movements (nystagmus), headache, sweating, ringing in the ears, hearing loss, visual disturbances. weakness, difficulty speaking, a decreased level of consciousness, and difficulty walking.

Methods described herein may be used to reduce or eliminate symptoms in individuals who are experiencing a vertigo episode. Individuals who are experiencing a vertigo episode may be identified as experiencing vertigo by a medical history and physical exam. CT scans, blood tests, magnetic resonance imaging (MRI), and electrocardiogram (ECG) may also be performed depending on the suspected cause. Physical examination often shows signs of abnormal eye movements, called nystagmus.

Such individuals who have been identified as experiencing vertigo may be treated with a device such as those set forth in U.S. Pat. Nos. 4,981,146, 6,076,018, 6,567,695, 6,735,480, 7,127,288, 7,171,266, 7,171,276, 7,983,761, or provisional application No. 62/614,212 entitled WEARABLE DEVICE FOR DELIVERING ELECTRICAL STIMULATION TO THE P6 ACUPUNCTURE POINT and filed Jan. 5, 2017. Such devices are used to apply electrical stimulation to the ventral side of the wrist (or the P6 acupuncture point or the median nerve). Any suitable means of applying electrical stimulation to the median nerve should work to relieve and eliminate vertigo symptoms in an individual suffering from vertigo. Also, while the method generally uses the electrical stimulation to the median nerve at its superficial route through the wrist, stimulation a the median nerve along its entire course should provide similar benefits.

In some embodiments, the P6 electrostimulation device provided herein the pulse generation (output) of the device is as follows. Waveform may be asymmetrical balanced and biphasic. In some embodiments, the power level of the output is from 0.1 mA to 50mA. In some embodiments, the power level of the output is from <1 mA to 40 mA. In some embodiments, ten (10) intensity levels (defined by the maximum current amplitude measured at the peak of the stimulus pulse when unit is driving a 500-ohm purely resistive load) will be available (all expressed as “setting level=output in milliamperes”) are provided. In some embodiments, the ten intensity settings may be as follows: 1=8, 2=11, 3=14, 4=17, 5=20, 6 =24, 7=28, 8=32, 9=36, 10=40, at tolerances of ±15%. In some embodiments, amplitude to be nominally constant current (+/−20% of output into 500-ohm load), is tested at 200 ohm and 1000 ohm loads. In some embodiments, amplitude may be automatically modulated using a 1 second ramp up to setting, 2 seconds constant at setting, 1 second ramp down to zero or lowest possible value, repeating continuously. In some embodiments, pulse width may be 350 μseconds +/−10%. In some embodiments, pulse frequency may be 31 Hz (32 msec period between alternating monophasic pulses) +/−10%. In some embodiments, pulse waveform may be such that the ratio of: [pulse amplitude at 50+/−2 μseconds after the start of the pulse] to [peak pulse amplitude at start of pulse] may be equal to 0.51+/−12%. In some embodiments, waveform will be such that the ratio of: [pulse amplitude at 100+−4 μseconds after the start of the pulse] to [peak pulse amplitude at start of pulse] will be equal to 0.26+/−20%. In some embodiments, the device may incorporate an output regulation feature such that the stimulation output amplitude does not vary more than 20% from the nominal as the battery charge decreases while in use. In some embodiments, the device may incorporate an automatic shut off feature such that when output regulation cannot be maintained for any setting, the device will shut off. In some embodiments, battery capacity and performance will deliver a minimum of 16 hours of continuous therapy from a full charge at intensity level 5 (which is half of the maximum output, or 20 mA output level) when tested using a 500-ohm purely resistive load.

In some embodiments, the overall waveform comprises a series of bipolar trapezoidal waveform that make low frequency pulses. The waveform is initiated at low power levels of about 1 to 2 volts and ramps up over a period of about 1 second to a maximum level of 17-18 volts, and is maintained for about 2 seconds, and then ramps down over a period of about 1 second to low power levels of about 1 to 2 volts. (The voltage specified is measured across a 500-ohm resistive load as suggested in ANSI/AAMI NS-4 (1986). American National Standard for Transcutaneous Electrical Nerve Stimulators.) The individual pulses are separated by about 31-32 milliseconds (msec) (measured peak to peak), and last about 350 microseconds (μsec) at power levels of about 1-100 milliamps peak pulse height, in some embodiments preferably 10-50 milliamps peak pulse height and in some embodiments 10-35 milliamps peak pulse height. A wide range of pulse patterns may be used in noninvasive nerve stimulation devices. The individual pulses alternate between negative and positive pulses, and are said to constitute a bipolar waveform. The individual pulse is made of a sharply vertical spike which decays exponentially over a period of about 350 μsec, thus comprising a basically vertical leading edge and an exponentially decaying trailing edge to each individual pulse. The following pulse will be shaped the same, except that it will be of negative voltage. The exponential nature of the individual pulse decay maximizes the high frequency components in the signal. These high frequency components contribute to a lessening of the skin impedance, in particular the capacitive components. This contributes to a higher level of current able to enter the deeper tissues. The power levels may be adjusted up or down to intensify the therapeutic effect of the device or lessen the sensation causes by the device, according to the preferences of individual users. The pulse rate within the waveform may be increased or decreased also.

To use one of the devices such as those set forth in U.S. Pat. Nos. 4,981,146, 6,076,018, 6,567,695, 6,735,480, 7,127,288, 7,171,266, 7,171,276, 7,983,761, or provisional application No. 62/614,212 entitled WEARABLE DEVICE FOR DELIVERING ELECTRICAL STIMULATION TO THE P6 ACUPUNCTURE POINT and filed Jan. 5, 2017, to alleviate vertigo symptoms in a person suffering from vertigo, the user merely secures the device on their wrist positioning the electrodes over the P6 acupuncture point, in electrical contact with the skin overlying the median nerve. The user then turns the device on, adjusts it to a comfortable power level, and allows stimulation to continue for a few minutes, for example 5-10 minute or longer, in some embodiments 30 minutes of longer, in some embodiments 30 minutes of longer, in some embodiments 90 minutes of longer, in some embodiments 120 minutes of longer, in some embodiments 150 minutes of longer, in some embodiments 180 minutes of longer, in some embodiments longer than 180 minutes, to achieve relief. The device may be applied intermittently, once every hour or so, or continuously. The device provides electrical current and voltage to the electrodes to relieve symptoms of vertigo in a person experiencing vertigo symptoms. The device provides electrical current and voltage to the electrodes to prevent or reduce symptoms of vertigo in a person with an underlying condition that causes vertigo and vertigo symptoms. The device will eliminate the symptoms entirely, or reduce them to more tolerable levels.

The extent that an individual suffering from vertigo is provided with relief from vertigo symptoms, such relief may be complete or a reduction in severity. The relief of symptoms may extend to all symptoms or a subset. In some embodiments, the method of treatment to an individual suffering from vertigo may result in reduction or elimination of one or more vertigo symptoms including feeling as if one is spinning, tilting, swaying, or being unbalanced or pulled to one direction, vomiting, abnormal or jerking eye movements (nystagmus), headache, sweating, ringing in the ears, hearing loss, visual disturbances, weakness, difficulty speaking, a decreased level of consciousness, or difficulty walking. Additionally, the methods may reduce or eliminate nausea caused by vertigo.

In a preferred embodiment, the P6 electrostimulation devices comprises pair of metal electrodes made of 316L stainless steel manufactured using metal injection molding technology that are substantially D-shaped, i.e. having a straight edge and an arcuate edge. The D shaped electrodes made of 316L, stainless steel manufactured using metal injection molding technology are arranged such that when the device is properly worn by a user and the electrodes are in contact with the P6 point on a user's wrist, the straight edges of the electrodes oppose each other, i.e. arranged with the straight edges facing each other in apposition. The straight edges are thus parallel to each other. The electrodes are arranged on the device such that when the device is properly worn by a user and the electrodes are in contact with the P6 point on a user's wrist, the straight edges are perpendicular with the direction of the user's arm between the wrist and elbow. The straight edges have a length of about 1 inch, and the arcuate edges having an inner radius of about 0.5 inches. in a preferred embodiment, D shaped electrode is about 22.00 to about 25.50 mm and the arcuate edges having an inner radius of about 10.50 to about 13.00 mm. In a preferred embodiment, each D shaped electrode has a thickness of about 0.50 to about 1.75 mm. In a preferred embodiment, D shaped electrode is about 23.00 to about 24.50 mm and the arcuate edges having an inner radius of about 11.00 to about 12.70 mm. In a preferred embodiment, each D shaped electrode has a thickness of about 0.80 to about 1.40 mm. In a preferred embodiment, D shaped electrode is about 23.84+/−0.10 mm and the arcuate edges having an inner radius of about 11.83 +/−0.10 mm. in a preferred embodiment, each D shaped electrode has a thickness of about 1.08+/−0.1.0 mm.

The electrodes are arranged on the device such that the straight edges of the electrodes face each other in apposition and are separated by a gap. In some embodiments, the gap is 0.05 to 0.5 inches or 1-15 mm. In some embodiments, the gap is 0.1 to 0.25 inches or 2-7.5 mm. In some embodiments, the gap is 3-5 mm. In some embodiments, the gap is 0.14 inches.

In some embodiments, the D shaped electrodes are used in the P6 electrostimulation devices and the electrodes are connected to the electrical conduits that supply electrical output at a position on or immediately adjacent to, i.e. within 5 mm, preferably within 3 mm of the center point of the arcuate edge.

The extent that an individual is provided with relief from vertigo symptoms, such relief may be complete or a reduction in severity. The relief of symptoms may extend to all symptoms or a subset.

While the preferred embodiments of the methods have been. described in reference to the environment in which they were developed, they are merely illustrative of the principles of the inventions. Other embodiments and configurations may be devised without departing from the spirit of the inventions and the scope of the appended claims.

1. A method of reducing or eliminating vertigo symptoms in an individual experiencing vertigo, said method comprising the steps of:
positioning a non-invasive nerve stimulation device on the individual's wrist, wherein the electrodes of the non-invasive nerve stimulation device are in electrically conductive contact with skin at acupuncture point P6 of the wrist;
generating a stimulation signal; and
delivering the stimulation signal to the acupuncture point P6 of the wrist to reduce or eliminate one or more vertigo symptoms in the individual;
wherein the vertigo symptoms are selected from the group consisting of: feeling of spinning, tilting, swaying, or being unbalanced or pulled to one direction, vomiting, abnormal or jerking eye movements (nystagmus), headache, sweating, ringing in the ears, hearing loss, visual disturbances, weakness, difficulty speaking, a decreased level of consciousness, and difficulty walking.

2. The method of claim 1, wherein the stimulation signal comprises individual pulses at power levels of about 1-100 milliamps peak pulse height, at a pulse frequency of about 31 Hz+/−10% such that there is a period of 32 msec between alternating monophasic pulses.

3. The method of claim 2, wherein the stimulation signal comprises individual pulses at power levels of about 1-50 milliamps peak pulse height at tolerances of ±15%.

4. The method of claim 2, wherein the stimulation signal comprises individual pulses at power levels of about 1-40 milliamps peak pulse height at tolerances of ±15%.

2. The method of claim 2, wherein. the stimulation. signal comprises individual pulses at power levels of about 8-40 milliamps peak pulse height at tolerances of ±15%.

6. The method of claim 2, wherein the stimulation signal comprises individual pulses at power levels selected from the group consisting of: about 8 milliamps peak pulse height at tolerance of ±15%; about 11 milliamps peak pulse height at tolerance of ±15%; about 14 milliamps peak pulse height at tolerance of ±15%; about 17 milliamps peak pulse height at tolerance of ±15%; about 20 milliamps peak pulse height at tolerance of ±15%; about 24 milliamps peak pulse height at tolerance of ±15%; about 28 milliamps peak pulse height at tolerance of ±15%; about 32 milliamps peak pulse height at tolerance of ±15%; about 36 milliamps peak pulse height at tolerance of ±15%; and about 40 milliamps peak pulse height at tolerance of ±15%.

7. The method of claim 1, wherein the stimulation signal is delivered for 5-10 minutes or longer.

8. The method of claim 1, wherein the stimulation signal is delivered for 30 minutes or longer.

9. The method of claim 1, Wherein the stimulation signal is delivered for 60 minutes or longer.

10. The method of claim 1, wherein the stimulation signal is delivered for 90 minutes or longer.

11. The method of claim 1, wherein the stimulation signal is delivered for 120 minutes or longer.

12. The method of claim 1, Wherein the stimulation signal is delivered for 150 minutes or longer.

13. The method of claim 1, wherein the stimulation signal is delivered for 180 minutes or longer.

14. The method of claim 1, wherein the stimulation signal is delivered for longer than 180 minutes.

15. A method of preventing or reducing vertigo symptoms in an individual identified as being susceptible to vertigo, said method comprising the steps of:
positioning a non-invasive nerve stimulation device on the individual's wrist, wherein the electrodes of the non-invasive nerve stimulation device are in electrically conductive contact with skin at acupuncture point P6 of the wrist;
generating a stimulation signal; and
delivering the stimulation signal to the acupuncture point P6 of the wrist to reduce or eliminate one or more vertigo symptoms in the individual;
wherein the vertigo symptoms are selected from the group consisting of: feeling of spinning, tilting, swaying, or being unbalanced or pulled to one direction, vomiting, abnormal or jerking eye movements (nystagmus), headache, sweating, ringing in the ears, hearing loss, visual disturbances, weakness, difficulty speaking, a decreased level of consciousness, and difficulty walking.

16. The method of claim 15, wherein the stimulation signal comprises individual pulses at power levels of about 1-100 milliamps peak pulse height, at a pulse frequency of about 31 Hz +/−10% such that there is a period of 32 msec between alternating monophasic pulses.

17. The method of claim 16, wherein the stimulation signal comprises individual pulses at power levels of about 1-50 milliamps peak pulse height at tolerances of ±15%.

18. The method of claim 16, wherein the stimulation signal comprises individual pulses at power levels of about 1-40 milliamps peak pulse height at tolerances of ±15%.

19. The method of claim 16, wherein the stimulation signal comprises individual pulses at power levels of about 8-40 milliamps peak pulse height at tolerances of ±15%.

20. The method of claim 16, wherein the stimulation signal comprises individual pulses at power levels selected from the group consisting of: about 8 milliamps peak pulse height at tolerance of ±15%; about 11 milliamps peak pulse height at tolerance of ±15%; about 14 milliamps peak puke height at tolerance of ±15%; about 17 milliamps peak pulse height at tolerance of ±15%; about 20 milliamps peak pulse height at tolerance of ±15%; about 24 milliamps peak pulse height at tolerance of ±15%; about 28 milliamps peak pulse height at tolerance of ±15%; about 32 milliamps peak pulse height at tolerance of ±15%; about 36 milliamps peak pulse height at tolerance of ±15%; and about 40 milliamps peak pulse height at tolerance of ±15%.
